(19) United States
(12) Patent Application Publication
(10) Pub. No.: US 2025/0104873 A1
(43) Pub. Date: Mar. 27, 2025

(54) METHOD AND SYSTEM FOR NEUROPSYCHOLOGICAL PERFORMANCE TEST

(71) Applicants: Frederic Andre JUMELLE, Hong Kong (HK); Yat Wan LUI, Hong Kong (HK)

(72) Inventor: Frederic Andre JUMELLE, Hong Kong (HK)

(21) Appl. No.: 17/625,568

(22) PCT Filed: Jul. 9, 2019
(86) PCT No.: PCT/CN2019/095325

§ 371 Date: Jan. 7, 2022

(51) Int. Cl. G16H 50/30; G06N 3/0499; G06N 3/084; G06N 3/09

(52) U.S. Cl. CPC G16H 50/30; G06N 3/0499; G06N 3/084; G06N 3/09

(57) ABSTRACT

The present invention relates to a method and system for neuropsychological performance test, comprising A terminal device (101), used to interact with a cloud server (102) which stores user information and is logged into by user through the terminal device (101); the user information obtained by the terminal device (101) are input and stored to the cloud server (102) in a login state; a test module (400) comprises the user information, which is stored in the cloud server (102) or can be downloaded from the cloud server (102), and is directly accessed through said terminal device (101) and is trained by artificial neural network; and said user information comprises user biometrics or emotions identification information, neuropsychological performance test answers information, and user latency or user chronometrics information; and said terminal device (101) displays neuropsychological performance test results.

The present invention relates to a method and system for neuropsychological performance test. More specifically, the invention relates to a method and system for neuropsychological performance test based on cognitive neuroscience. And particularly the present invention relates to a method and system for performance test using a temperament inventory based on cognitive neuroscience which refers to the automatic emotional responses to experience that is moderately heritable and relatively stable throughout life—in order to obtain an accurate neuropsychological performance of a test subject or user.

### BACKGROUND

Cognitive neuroscience is a hybrid branch of the cognitive psychology and neuroscience or cognitive science. Based on the theory of cognitive neuroscience and experimental neuropsychology, neurolinguistics and computer models, the relationship between the psychological phenomenon of the subject and the brain structure is determined. The investigation techniques based on experimental cognitive neuroscience include transcranial magnetic stimulation, functional magnetic resonance imaging, electroencephalography, and magnetoencephalography. Other brain imaging techniques, such as positron tomography and single-photon computed tomography, are sometimes used. Single cell potential recording is used on animals and further brought out the compelling evidence. Other techniques used for investigation could be micro-neurograms, EMG on the face, and eye trackers. Applied neuroscience has been integrating research results from different fields and on different scales enough to reach a unified descriptive model of the brain functioning in regard to the biosocial personality.

With the development of cognitive neuroscience, Professor Robert Cloninger proposed a unified theory of biological social personality. He believes that the method of obtaining accurate neuropsychological performance not only needs to consider behavioral factors, but also needs to consider potential biological and social determinants—and distinguish between the perceptual and the conceptual factors. The Temperament and Character Inventory (TCI) is based on the above theory. It aims to distinguish the hereditary nature (Temperament) from the acquired nature (Character) of the personality development through an experimental method (Inventory) to obtain the subject's neuropsychological performance. TCI can also be used to identify various personality disorders to examine the extent of personality disorder development. The TCI has seven dimensions, four of which are dimensions of the Temperament: Novelty Seeking (NS), Harm Avoidance (HA), Reward Dependence (RD) and Persistence (PS); and the three others are dimensions of the Character: Self-Directiveness (SD), Cooperativeness (C) and Self-Transcendence (ST). In the prior art, the Temperament and Character Inventory revised version (TCI-R) is used to evaluate the psychological status of the subject through a combination of the personal characteristics: the biological characteristics of the subject such as physical health factors, genetic vulnerability, addictive behaviors; the social characteristics such as family environment, close relationships, marital status; and the psychological characteristics such as cooperative skills, social skills, relational skills, self-esteem and mental health. However, the traditional TCI-R approach is more about self-management rather than self-report which makes it a relatively biased intervention that obviously misses the emotional assessment.

Neuropsychology is the study and characterization of the behavioral modifications that follow a neurological trauma or condition. It is both an experimental and clinical field of psychology that aims to understand how behavior and cognition are influenced by brain functioning and is concerned with the diagnosis and treatment of behavioral and cognitive effects of neurological disorders.

In another register of assessment, the Rorschach or Inkblot test in the prior art is a projective personality test which allows the test subject to establish connections to his inner imaginary world through a certain medium, revealing his personality in an unconstrained manner. It is a pure personality test method based on psychology. This method is usually used to approach the psychology of high-level managers and criminals with sophisticated mind. This test involves emotional assessment, but the results obtained are inevitably biased.

In another reference in the prior art, the development of neuroeconomics and neurofinance in particular has described decision-making and psychology as closely intertwined and therefore isolated strong trends in this field. For example, Professor Daniel Kahneman has conducted research on the human decision-making process in uncertain situations and has proven that human behaviors are systematically biased by irrational emotions and lead to decisions at the opposite corner of the best economic outcome possible, so can eventually accelerate the outburst of financial crisis. These research results have led to express the theory of a rational investor who could be freed from the emotion response and take rational decision even under pressure. Based on the above background of theoretical disciplines and the continuous advancement of technology, such as the development of artificial intelligence and artificial neural network technologies, a system and method combining neuroscience with artificial intelligence technology is needed to promote the adoption of a more rational profiling test. The psychology of the test subject must be analyzed and processed to obtain an accurate psychological profile result in order to apply this result to a number of scenarios that require accurate neuropsychological performance test results, such as screening, recruitment, regulatory onboarding, digital tracking, fraud forensics and all remote and/or virtual services that do not necessarily require a face-to-face meeting.

BRIEF SUMMARY The invention provides a test method and system based on cognitive neuroscience to obtain an accurate neuropsychological performance test result of a test subject or user.

The invention provides a system for neuropsychological performance test, comprising: a terminal device (101), used to interact with a cloud server (102) which stores user information and is logged into by the user through the terminal device (101); the user information obtained by the terminal device (101) are input and stored to the cloud server (102) in a login state; a test module (400) comprises the user information, which is stored in the cloud server (102) or can be downloaded from the cloud server (102), and is directly accessed through said terminal device (101) and is trained by artificial neural network; and said user information comprises user biometrics or emotions identification information, neuropsychological performance test answers information, and user latency or user chronometric information; and said terminal device (101) displays neuropsychological performance test results.

The present invention also provides a method to conduct the neuropsychological performance test, comprising: using a terminal device (101) to interact with a cloud server (102) which stores user information and is logged into by user through the terminal device (101); inputting and storing the user information obtained by the terminal device (101) to the cloud server (102) in a login state; Accessing a test module (400) comprising the user information directly through said terminal device (101), which is stored in the cloud server (102) or can be downloaded from the cloud server (102), and training said test module (400) by artificial neural network; and said user information comprises user biometrics or emotions identification information, neuropsychological performance test answers information, and user latency or user chronometric information; and displaying neuropsychological performance test results by said terminal device (101).

The present invention can be applied to customer profiling, neuropsychological performance test, and also be applied to pre-screening, remote screening and onboarding of human resources; sorting of personal accounts, fraud prevention and forensics for social media; matchmaking in client relation management and dating; onboarding and remote onboarding of new customers in the financial services industry in compliance to Know Your Client (“KYC”) or Customer Due Diligence (“CDD”) regulations and also any new virtual services to persons including providing smart ID for the smart cities etc. Compatible with other identification and identity authentication/verification technologies, the present invention allows the creation of true personal identity by using personal metrics with a high degree of accuracy and security. So that, some of the biggest challenges of internet nowadays such as an outrageous amount of fake/ghost accounts, in particular fake social media accounts pose a threat to the society and they could also be dealt with through the present invention.

### BRIEF DESCRIPTION OF THE SEVERAL VIEWS OF THE DRAWINGS

The drawings referenced herein form a part of the specification. Features shown in the drawings are meant as illustrative of only some embodiments of the invention, and not of all embodiments of the invention unless otherwise explicitly indicated.

FIG. 1 is a schematic diagram of a neuropsychological performance testing system 100 including a test module of the present invention.

FIG. 2 is a schematic structural view of a terminal device of the present invention.

FIG. 3 is a flow chart showing the operation of the neuropsychological performance testing system of the present invention.

FIG. 4 is a block diagram showing the working principle of the test module 400 of the present invention.

FIGS. 5(a)-(d) are schematic illustrations of examples of time responses of different tests and results in the neuropsychological performance testing system of the present invention, respectively.

FIG. 6(a)-(b) are schematic diagrams of an artificial neural network of a neuropsychological performance testing system of the present invention.

FIG. 7(a) is a flow chart of the neuropsychological performance testing 100 of the present invention.

FIGS. 7(b)-(f) are schematic diagrams of screen shots corresponding to the flow shown in FIG. 7(a).

FIG. 8 is a diagram showing an example of an original result of the neuropsychological performance testing system of the present invention.

FIG. 9 is a demographic data or proxies (in profiling) of an example of the subject shown in FIG. 8.

FIG. 10 is the purchase-order data of the end user (client) corresponding to the ideal subject of the search.

FIG. 11 is an illustration of the various parameters of the present invention.

FIG. 12 is a representation of possible ranges of results for testing using the present invention.

FIG. 13 is a view showing a comprehensive analysis chart of test results obtained by the present invention.

FIGS. 14(a) and 14(b) are explanatory results of analysis using the test results of the present invention.

FIG. 15 is a table of High Processing Performance & Flexibility of the present invention.

### DETAILED DESCRIPTION

It is understood that the components, as generally described and illustrated in the Figures herein, may be arranged and designed in a wide variety of configurations. Thus, the following detailed description of the embodiments of the apparatus, system, and method, as presented in the Figures, is not intended to limit the scope of the invention, as claimed, but is merely representative of selected embodiments.

The functional unit described in this specification with elements labeled as managers. A manager may be implemented in programmable hardware devices such as field programmable gate arrays, programmable array logic, programmable logic devices, or the like. The manager may also be implemented in software for execution by various types of processors. An identified manager of executable code may, for instance, comprise one or more physical or logical blocks of computer instructions which may, for instance, be organized as an object, procedure, function, or other construct. Nevertheless, the executables of an identified manager need not be physically located together but may comprise disparate instructions stored in different locations which, when joined logically together, comprise the manager and achieve the stated purpose of the manager.

Indeed, a manager of executable code could be a single instruction, or many instructions, and may even be distributed over several different code segments, among different applications, and across several memory devices. Similarly, operational data may be identified and illustrated herein within the manager and may be embodied in any suitable form and organized within any suitable type of data structure. The operational data may be collected as a single data set or may be distributed over different locations including over different storage devices, and may exist, at least partially, as electronic signals on a system or network.

Reference throughout this specification to “a select embodiment,” “one embodiment,” or “an embodiment” means that a particular feature, structure, or characteristic described in connection with the embodiment is included in at least one embodiment. Thus, appearances of the phrases “a select embodiment,” “in one embodiment,” or “in an embodiment” in various places throughout this specification are not necessarily referring to the same embodiment.

Furthermore, the described features, structures, or characteristics may be combined in any suitable manner in one or more embodiments. In the following description, numerous specific details are provided, such as examples of recovery manager, authentication module, etc., to provide a thorough understanding of embodiments. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

The illustrated embodiments will be best understood by reference to the drawings, wherein like parts are designated by like numerals throughout. The following description is intended only by way of example, and simply illustrates certain selected embodiments of devices, systems, and processes that are consistent with the invention as claimed herein.

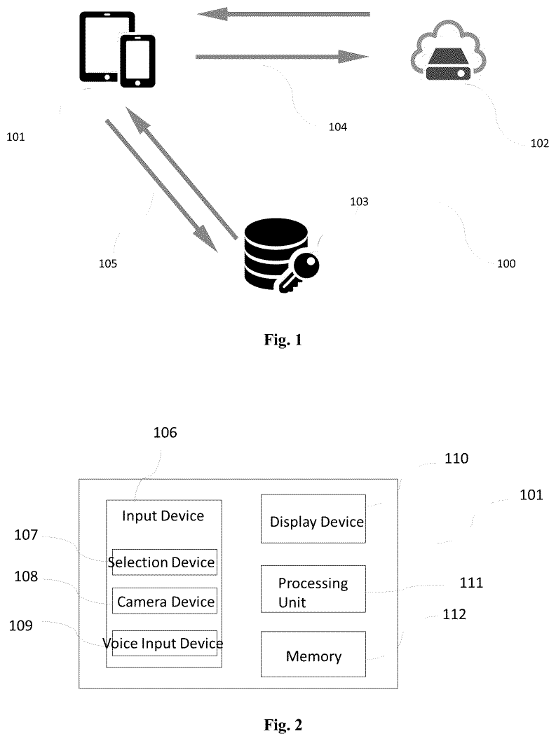

FIG. 1 is a schematic diagram of a neuropsychological performance testing system 100 including a test module of the present invention. The terminal device 101 is an end user input interface and may be a mobile communication device or a tablet computer or other mobile terminal including a wireless communication device. The terminal device 101 is mainly used to interact with the cloud server 102, and the cloud server 102 stores user information. The user logs into the cloud server 102 through the terminal 101, and inputs and stores the user video, voice and other index obtained by the terminal device 101 to the cloud server 102 in the login state. The local database 103 is stored in the application program. In the local database 103, the questionnaire is fully encrypted and randomly provided to the end user by local model processing to appear on the terminal device 101 screen, and based on the end user facial expression information obtained by the terminal device 101, combined with the input of the user for each question of the questionnaire, the neuropsychological performance result is given. The test module or questionnaire (not shown in FIG. 1) may be included in the cloud server 102 and the related information is directly accessed through the terminal device 101. Alternatively, the test module can be downloaded from the cloud server 102 or the local database 103 to the terminal device 101 at step 104 or step 105. See FIG. 2 for a detailed description of the test module.

FIG. 2 is a schematic structural view of a terminal device of the present invention. The terminal device 101 comprises an input device 106 comprising a questionnaire selection device 107, such as a touch button; and/or an imaging device 108, such as a camera, capable of capturing the facial expression of the end user; and/or a voice input device 109, for example a microphone. The terminal device 101 further includes a display device 110, a processing unit 111, and a memory 112. The terminal device 101 obtains information that the end user responds to the questionnaire through the input device 106, stores the information to the cloud server 102, and inputs it into the local database 103; through the analysis located in the cloud server 102 or the local database 103, through the display device 108 displays the results of the neuropsychological performance.

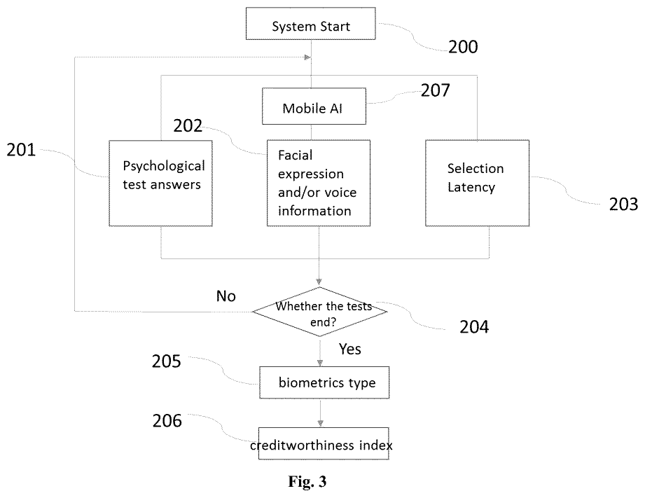

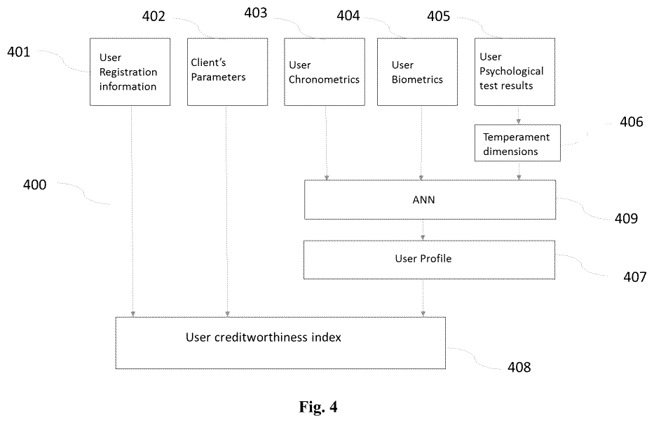

FIG. 3 is a flow chart showing the operation of the neuropsychological performance testing system of the present invention. At step 200, the neuropsychological performance testing system begins to run. At step 201, the subject inputs the selection of answers to the questionnaire selection device 107. At step 202, the subject's facial expression information for neuropsychological performance is obtained by the camera device 108 and/or the subject's voice information for neuropsychological performance is obtained by the voice input device or microphone 109, for example a laughter or a change in the voice pitch. At step 203, the processing unit 111 of the terminal device records the delay time (latency) of the subject's input of the question answer. And, at step 204, it determines whether the performance test has ended. After the test is finished, at step 205, the analysis module is used to obtain the neuropsychological performance of the test subject. If the test has not ended, the above steps 201, 202 and 203 are repeated; Finally, on the basis of the neuropsychological performance, the psychological credit index of the subject is obtained at step 206. At step 203, the facial expression information and the delay time (latency) information are input into the analyzing device; the analyzing device analyzes the neuropsychological performance of the test subject, and the output psychological test result is directed to the calculation module, and the trustworthiness (TWI) or alternatively creditworthiness index (CWI) of the subject is calculated by performing the following formula (1) to obtain the result of this analysis using a mobile artificial intelligence and an artificial neural network (“ANN”):

$$Psychological trustworthiness index TWI = [ ( RP \times T ) \times TT \times ( BT \times C ) ] \qquad ( 1 )$$

The result is in the scope of 0-120.

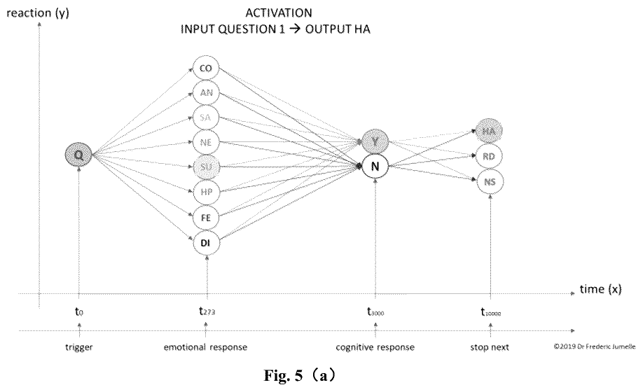

Among them, RP is a Risk Profile, T is Truthfulness, TT is a Thinking Type, BT is a Biometric Type and C is a Confidence score. The details will be described in FIG. 4.

FIG. 4 is a block diagram showing the working principle of the test module 400 of the present invention. The test module of the present invention is a five-part information input test module, and the first part is the user registration information or proxies 401 of the test subject, including: age, gender, job level, education level, genetic heritage, region, portable device, browser or username, IP address or phone number, date, time, weather at the time of the survey; the second part is the organization (end client's parameters) data 402 who needs the neuropsychological performance assessment for a specific customer search or for a matchmaking process in CRM, including: client manager risk profile, client industry type, cohort risk profile median, search user risk profile, search user education level, user target job/investment level, user target job/investor role, specific requirements, preferred user TWI/CWI index range, date; the third part is the latency (user chronometric) information 403 on the test user, including: average test latency, minimum/maximum/average response time, consistency/variability, decisiveness/uncertainty, cadence/exceptions; the fourth part is the user biometrics (emotions) identification information 404, including: facial emotions, consistency/variability, duration/transition, contradiction/unusual reactions, situation awareness, lie detection by identification of inconsistencies.

In addition to the above four parts, a fifth part refers to neuropsychological performance profiling test results 405, including the answers to the assessment questions; The assessment questions of the questionnaire are generally YES or NO questions, in the number of 30 per questionnaire, which are translated and localized according to linguistic and semantic requirements of the language of the user, and the social and cultural background of the user region. The goal of the psychological assessment is to obtain the temperament dimensions information 406 of the test subject, furthermore, is combining them with the latency (time) information 403 of the subject and the biometric (emotion) identification information 404 of the subject to obtain the user performance 407 of the subject. Finally, the subject's trustworthiness or creditworthiness index 408 can be obtained by combining the user registration information 401 with the user performance data 407. Matchmaking for CRM (Client Relation Management) can be achieved by combining the User performance data 407 with the Client's Parameters 402 for which psychological testing of the client representative is required.

The method to obtain the above temperament dimensions information 406 is aligned with the prior art of Temperament and Character Inventory revised version (TCI-R). Four of TCI-R dimensions are temperament related dimensions: including Novelty Seeking (NS), Harm Avoidance (HA), Reward Dependence (RD) and Persistence (P); and three are character related dimensions: including Self-Directedness (SD), Cooperativeness (CO) and Self-Transcendence (ST). According to Prof Cloninger, Temperament related dimensions are relatively stable throughout life while Character related dimensions are more progressive and variable throughout life. Among the 7 dimensions, sub-dimensions have been identified for Novelty Seeking (NS) including Exploratory Excitability (NS1), Impulsiveness (NS2), Extravagance (NS3), Disorderliness (NS4); for Harm avoidance (HA) including Anticipatory worry (HA1), Fear of uncertainty (HA2), Shyness with strangers (HA3), Fatigability (HA4); for Reward Dependence (RD) including Sentimentality (RD1), Openness to warm communication (RD2), Attachment (RD3), Dependence (RD4); for Persistence (PS) including Eagerness of effort (PS1), Work hardened (PS2), Ambitious (PS3), Perfectionist (PS4); for Self-Directedness (SD) including Responsibility (SD1), Purposefulness (SD2), Resourcefulness (SD3), Self-acceptance (SD4), Enlightened second nature (SD5); for Cooperativeness (C) including Social Acceptance (C1), Empathy (C2), Helpfulness (C3), Compassion (C4), Pure-hearted Conscience (C5); for Self-Transcendence (ST) including Self-forgetfulness (ST1), Transpersonal identification (ST2), Spiritual acceptance (ST3). Amongst the above described 7 dimensions, the present invention mainly relates to NS, HA and RD dimensions because they relate more clearly to a genetic heritage and appear to be more objective as a criterion of judgment for accurate performance. These dimensions have been investigated by functional MRI and correlated to the neurophysiology of the brain that is using different circuits for conveying messages with various biochemicals involved to enable the transmission between natural neurons also called the ‘Neurotransmitters’. Among them, NS is associated with low Dopaminergic activity related to the Dopamine; HA is associated with high Serotoninergic activity related to the Serotonin; and RD is associated with low Adrenergic activity related to the Norepinephrine and also with a dysfunctional endocannabinoid system.

The test module of the present invention is in the form of a questionnaire of 30 Yes or No questions. The type and nature of the questions are variable. The structure of the questionnaire can be modulated, standardized or personalized according to the rules to access the local database.

Each of these questions is corresponding to a pair (two) of answers whether Yes or No that leads to an interpretation of the temperament type of the test subject. The questions are called bijective because of this specific structure.

Besides the bijective nature of the questions, they are also separated in two groups the major types and the minor types. The major types are related to main and obvious situations or issues in life while the minor types are related to more complex ideas and problems. In the invention questionnaire, the major type questions are few times more frequent than the minor types.

For example, the following questions are major types:
Do you like gardening? HA/NS
Do you like dancing? RD/HA
Do you seek revenge after being hurt? NS/RD

Each question has its specific bi-dimensional properties. As noted above, the first question: “Do you like gardening?” is a major type of question related to Harm Avoidance (HA) versus Novelty Seeking (NS). When the answer to this question is Yes, it means that the temperament of the test subject or user is in favor of HA, to the contrary if the answer is No, the temperament would be in favor of NS. NS is for risk seeking (Taker) and HA is for risk aversion (Averse). A similar approach is applied to a RD/HA question type such as “Do you like to dance?”. If the user chooses to answer No, it would in favor of HA which is for risk aversion (Averse); if the user's response is Yes it would be in favor of RD which is for risk dependent (Dependent). Each answer to a question is granting one datapoint in one of the 3 dimensions/bins and the highest score of the 3 bins where the most datapoints are accumulated will determine the temperament result of the user also called the primary score of the risk profile. The second bin with the second highest number of datapoints will determine the secondary score of the risk profile and the combination of primary and secondary scores will become the user risk profile score.

In this invention the brain processing time is called latency and the performance test average latency information is the average time of the questionnaire test divided by the total number of questions asked or alternatively the sum of the time lapse between question until answer for each of the 30 questions, or more if revalidation is needed, and divided by this number of questions. The range of the test average latency is between 0.273 and 10 seconds. The result obtained by a test user is sorted and put into one of a three or a five categories model. In the three categories model, it can be described as short, median and long while timed in milliseconds or seconds. Short means less than 3 seconds, median means 3 to 5 seconds and long means more than 5 seconds assuming that the test population in which the test user belongs has a normal distribution and its median is around 3 seconds. For this reason, the five categories model is favored because it is more compatible to a 2 standard deviation model and it is divided into extra short (XS), short, median, long, extra long (XL). A brain processing time or latency of 0.273 to 2 seconds is regarded as extra short; 2 to 3 seconds is short; 3 to 5 seconds is median; 5 to 7 seconds is long; and extra long if it is more than 7 seconds. The first standard deviation is set between 2 to 7 seconds assuming the population is normal or quasi normal and the median of population is around 3 seconds. Any result below 2 or above 7 seconds is considered unusual and required a new question of similar nature for verification/revalidation of the questionnaire. If there are more than 6 revalidations, the whole questionnaire result is deemed untruthful therefore invalid. While the questionnaire is being activated, the camera 108 is starting to capture the video of the survey which is divided in 2 parts: the capture of the facial emotion information immediately after the reception of the question on the screen also called ‘reaction time’ which the median is 0.273 second in humans—and processed by the mobile AI for facial recognition; then the video capture continues during the reflection time until the response is decided by the test user whether by typing Yes or No on the screen or record a vocal answer or voice information through the voice input device 109 or both text and vocal if used for voice-movement coordination patterns study. The facial expression information, also called emotions, of the test subject are compared with the biometric information pre-stored in the system, also called training data. For the matched information, it will determine whether that the input is normal and the biometric type is corresponding to the expected emotion of the test subject; or if it deviates, how much is the deviation, and what is the deviation that would be sufficient to disqualify the record. If the mobile AI of the invention disqualifies an emotion to a question, a new question of the similar type will be asked again at the end of the 30 questions standard model and will make the questionnaire of 31 questions, 30 standard plus 1 revalidation that will contribute to assess the truthfulness. For example, for the question “Do you like gardening?”, the primary biotype is “Surprise” if the answer is expected to be ‘Yes’ and the secondary biotype is “Neutral” if the answer is expected to be ‘No’. The training data will modify the weights and biases of the ANN and test subject on real population will also allow the invention to build culture-based and regional repertoire of reactions and compare repertoires for a very precise performance result. It is presumptuous to offer a conclusion at this stage on whether the norm of population should be the median or the average. The backpropagation error calculation should also help lower the cost function of the invention multilayer perceptron, which will be discussed in detail below.

In addition to the major type of questions described above, the present invention questionnaire is using a set of minor type questions in lesser number of occurrences, such as:
Do you feel happy while spending money? NS/HA
Do you keep your problems to yourself? HA/RD
Do you consider right from wrong before taking decisions?? RD/NS

Alike the major types of question, each minor question has its own bi-dimensional properties referring to temperament characteristics. The temperament datapoint is related to the question bijective structure, in the case whether the test subject answers Yes or No to the question. The exact same processing of information of the major types applies to the minor types. Similarly, the present invention captures the facial emotion of the test subject in reaction to the question, records the latency time to process the question and eventually his voice information in response to the question. The correlation algorithm and the processing of normal and unusual reactions are considered as the core of the present invention leading to deliver a report of credibility and truthfulness on the test subject also called Creditworthiness Index or CWI and Trustworthiness Index or TWI.

For assessment purpose, the present invention refers to 8 different emotion types used by facial recognition systems including mobile AI systems for facial recognition on portable device, including the device recommended for taking the test of the invention. The 8 types are: Contempt (“CO”), Surprise (“SU”), Anger (“AN”), Sadness (“SA”), Neutral (“NE”), Disgust (“DI”), Fear (“FE”) and Happiness (“HP”). The present invention also attributes a certain coefficient to each emotion in order to derive scores and index through the formula.

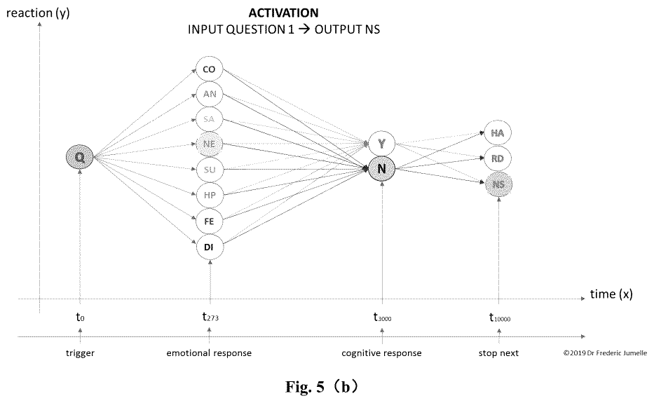

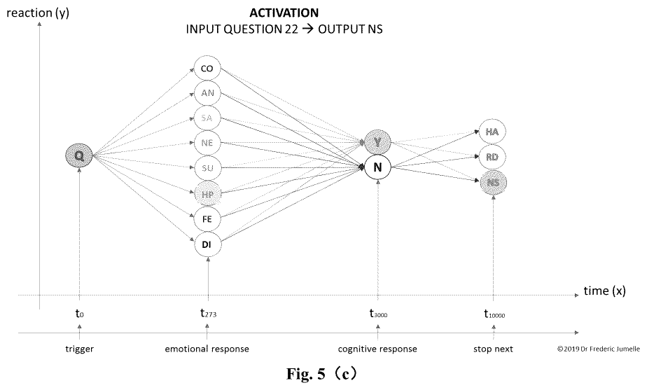

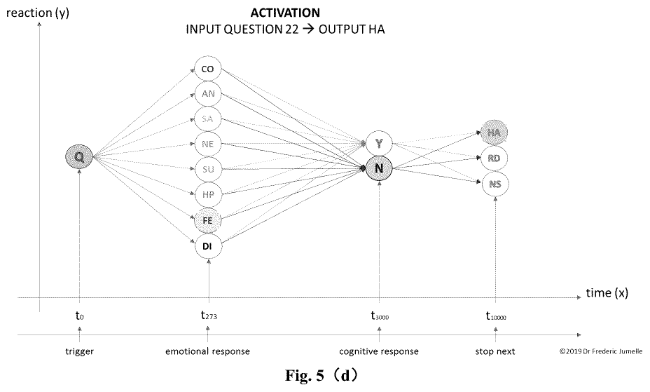

FIGS. 5(a)-(d) are schematic illustrations of 4 examples of answers to the 2 types of questions (major and minor) whether Yes or No is answered with a breakdown of the timeline between the trigger Q to the emotional response (1 choice among 8) to the cognitive response (Yes or No) and the stop of the chronometer until the next question appears on the screen. This single process is repeated 30 times or until the end of the test with a maximum of 36 questions in total of which only 30 will be accounted. FIG. 5(a)(b) shows that when receiving the major type question “Do you like gardening?”, the subject will likely express the corresponding emotion “SU” when answering ‘Yes’ or emotion “NE” when answering ‘No’, and that will determine whether HA or NS datapoint will go to the 3 bins result and accumulate until one of the bin contains more than the 2 others and therefore determine the primary risk profile. FIG. 5(c) (d) applies the same principle to the minor type question “Do you feel happy while spending money?”. Emotion is Happy for a Yes answer that makes a NS datapoint. Emotion is Fear for a No answer that makes a HA datapoint.

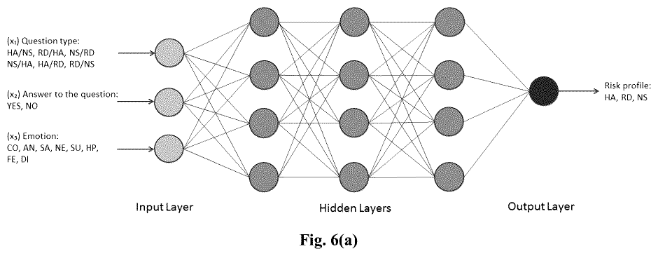

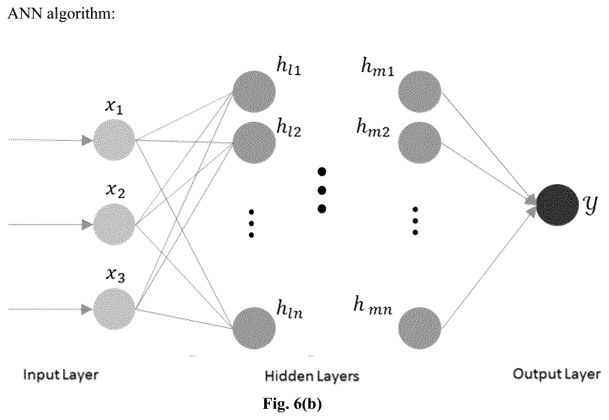

FIG. 6(a) is a schematic diagram of the feed forward artificial neural network also called a multilayer perceptron of a neuropsychological performance testing system of the present invention. The network is divided into three categories of layers of artificial neurons or nodes, the input layer, the hidden layers and the output layer. The input layer is a three-dimension vector for question type, answer, emotion. These 3 inputs are represented by integers. The question type (whether HA/NS, RD/HA, NS/RD, NS/HA, HA/RD, RD/NS) has integer value of 0, 1, 2, 3, 4 or 5, the answer type (whether Yes or No) value is 0 or 1, the emotion type whether Contempt (“CO”), Anger (“AN”), Sadness (“SA”), Neutral (“NE”), Surprise (“SU”), Happiness (“HP”), Fear (“FE”), Disgust (“DI”) has integer value from 0 to 7. Output is also an integer standing for one of the 3 risk profiles (whether HA, RD, NS) value is 0, 1 or 2.

For the training data, each entry consists of question type, answer, detected emotion. The ANN of the invention will start with a 3-layer model and feed the data into the model. After training, the optimal weights are obtained from which the nonlinear expression of the process can be achieved.

The Backpropagation is part of the training and consists of two phases: stimulus propagation and weight update. In the excitation propagation phase, the propagation link in each iteration consists of two steps: 1. Forward propagation phase: the training input is sent to the network to obtain the excitation response; 2. Back propagation phase: the excitation response corresponds to the training input. The target output is evaluated to obtain a response error of the output layer and the hidden layer. In the weight update phase, for each synapse (junction between nodes) weight, update is made as follows: 1. Multiply the input stimulus and response error to obtain a gradient of weights; 2. Multiply this gradient by a scale and invert it added to the weight; this ratio (percentage) will affect the speed and effect of the training process, thus becoming a “training factor.” The direction of the gradient indicates the direction in which the error is magnified, so it is necessary to reverse the weight when updating the weight, thereby reducing the error caused by the weight. Phases 1 and 2 can iterate through the iterations until the network's response to the input reaches a satisfactory predetermined target range. For example, if the question type is HA/NS and the user answers NO with a surprise (SU) face, the actual risk profile for that user is NS. Then the input is [0,1,4] and the model already knows that the output should be 2. If the output is 1, the model will modify the weight in the backward propagation, in particular multiply the weight by 2 the final result can be 2. FIG. 6(b) is a schematic diagram of an artificial neural network of a neuropsychological performance testing system of the present invention. FIG. 6(b) is an algorithm, wherein

x₁:question typeϵ[0,5]

x₂:answer to the questionϵ[0,1]

x₃: emotionϵ[0,7]

The proposed architecture is a supervised, fully connected, feed-forward artificial neural network. It uses back-propagation training and generalized delta rule learning.

### 1. Hyperparameters/Input & Output:

The input layer consists of three nodes, question type, answer to the question, and emotions, respectively. Question type is an integer from 0 to 5 (HA/NS, RD/HA, NS/RD, NS/HA, HA/RD, RD/NS). Answer is 0 or 1 (Yes or No). Emotions is from 0 to 7 (CO, AN, SA, NE, SU, HP, FE, DI).

The output layer consists of one node, risk profile. It is an integer from 0 to 2 (HA, RD, NS).

The number of hidden layers and the number of nodes in each hidden layer are determined through experiments of different combinations.

### 2. Training Process:

### 2.1. The Output of Each Node is:

$$𝒴 = \vec{w} x + \vec{b}$$

[symbol] is the weight of this node
[symbol] is the bias.

The weights and bias with random values are initialized.

The activation function is applied to the output of the node. In this case ReLU is applied, given by:

$$ϕ ( a ) = max ( 0 , a ) = max ( 0 , \vec{w} x + \vec{b} )$$

The output of output layer is given by summing up the output of all nodes in the last layer:

$$𝒴 = ϕ ( {\vec{w}}_{0} \cdot ( {\sum }_{1}^{N} {\vec{w}}_{h} x + {\vec{b}}_{h} ) + {\vec{b}}_{0} )$$

[symbol] ₕ is weight of last hidden layer
[symbol] ₕ is bias of last hidden layer
[symbol] ₀ and [symbol] ₀ are weight and bias of output layer

### 2.2. After the Output is Given, the Backpropagation is Run to Minimize the Error Between the Validation Datasets and the Corresponding Targets:

$${δ}_{h} = {𝒴}_{0} ( 1 - {𝒴}_{0} ) ( {𝒴}_{0} - t )$$

[symbol] ₀ is the output of output layer
t is the target output (ground truth)

### 2.3. For Each Node, Calculate the Back Propagation Error Term:

$${δ}_{h}^{′} = {𝒴}_{h}^{′} ( 1 - {𝒴}_{h}^{′} ) {\sum }_{1}^{N} {δ}_{h} \cdot {\vec{w}}_{h}$$

2.4. Update the Synaptic Weights from a Node in Layer n to a Node in Layer n+1, Given by:

$$Δ \vec{w} = - {𝒴δ}_{h}^{′} x$$

[symbol] is learning rate. Then:

$$\vec{w} = : \vec{w} + Δ \vec{w}$$

### 2.5. Calculate Mean Squared Errors:

$$MSE = \frac{1}{{N}_{ts}} \cdot {\sum }_{1}^{{N}_{ts}} {( {𝒴}_{0} - t )}^{2}$$

15% of the data is used for validation dataset.

### 2.6. Repeat the Forward Propagation and Back Propagation Until the Number of the Epoch Limit or Early Stopping Criteria is Reached.

After training, the model runs on 15% test dataset to calculate precision, accuracy and F score.

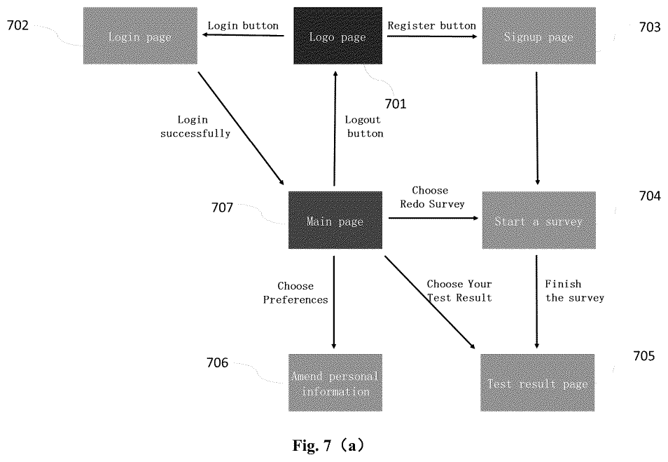

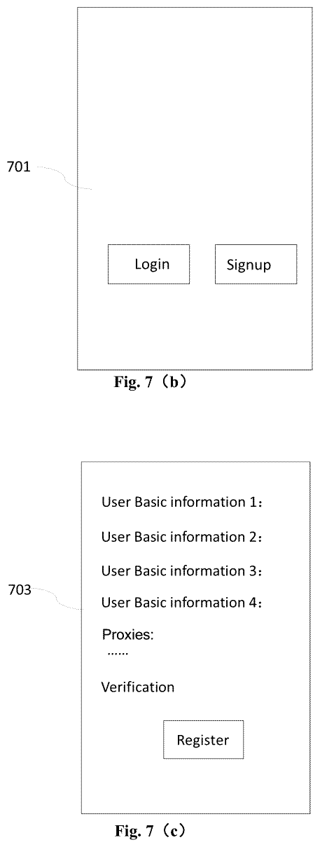

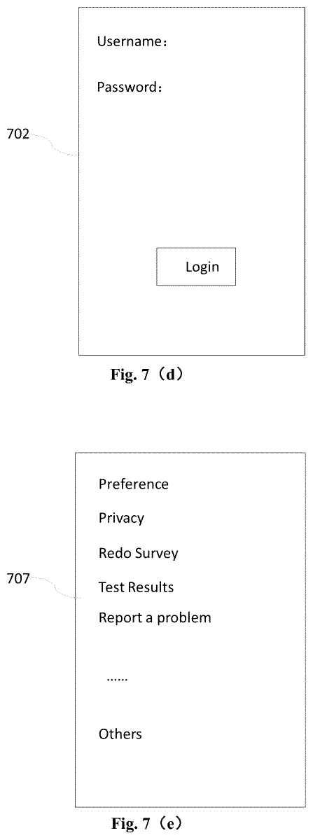

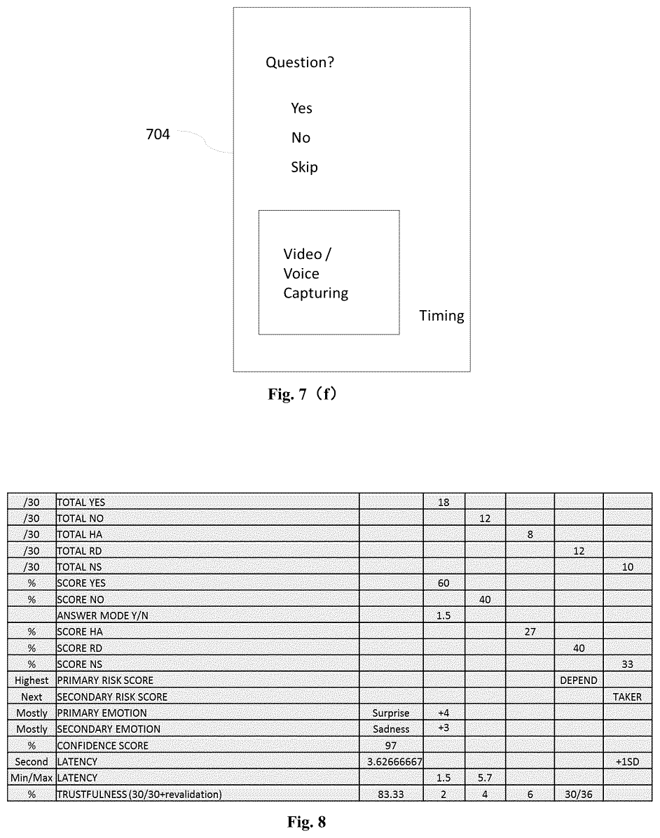

FIG. 7(a) is a flow chart of the neuropsychological performance testing system 100 of the present invention. FIGS. 7(b)-(f) are schematic diagrams of screen shots corresponding to the flow shown in FIG. 7(a). The trademark page interface 701 allows access to the login page interface 702 or to the signup (registration) page interface 703 respectively through the login key and the signup key; wherein the login page 702 relates to the authentication of the test subject by username and password; the signup page relates to the user basic information and proxies used for profiling of the test subject. Information and proxies such as age, gender, job level, education level, genetic heritage, region, portable device, browser or (sometimes) username, IP address or mobile number without country code, date of the test, time of the test, weather at the beginning of the test. The main page interface 707 is accessed through the login page 702. The main page interface 707 serves as a central interface articulating the subservience of the other modules, including the choices to: ‘redo survey’ at interface 704; check/pick (choose) the test result at interface 705; and choose to access preferences and amend/delete/cancel personal information (however non-sensitive) at interface 706. The start survey interface 704 allows the system to start the questionnaire of the neuropsychological performance test of the present invention while activating the camera and the microphone of the portable device after receiving the agreement of the test subject when requested at the end of the signup process at interface 703. During the survey at 704 and 705, the test subject interacts with the system at each stage until the result is released at 705 including the scores of risk profile, the thinking type and the biometric type and the index in the case of the search has been requested specifically by a client for the test subject under his consent.

FIG. 8 is a table showing an example of an original result of the neuropsychological performance testing system of the present invention. This diagram is for internal processing and will not be accessible to either the test subject or the client who may request for a search. It will be stored in a cloud archive with the video recording when the processing is completed. It shows the number of Yes and No responses, the number of answers in each dimension whether HA, RD or NS that and the percentage of each item out of the total. At the same time, it shows also the primary and secondary risk profile according to which of them reached the highest and second highest values of the percentage. The example given in FIG. 8 shows that the test subject's RD temperament dimension accounted for 40% and the NS accounted for 33% therefore the primary risk profile is ‘Dependent’ and the secondary is ‘Taker’. The test subject emotional type is primarily Surprise “SU” and secondary type is sadness “SA” with a confidence score of 97%. The average latency of the test for this subject is 3.6266 seconds while the median is set theoretically at 3. The test subject of this example contains 2 unusual latencies of less than 2 seconds and 4 unusual emotions which makes 6 revalidations in total therefore the trustworthiness score is 30/36 equals 83.33%.

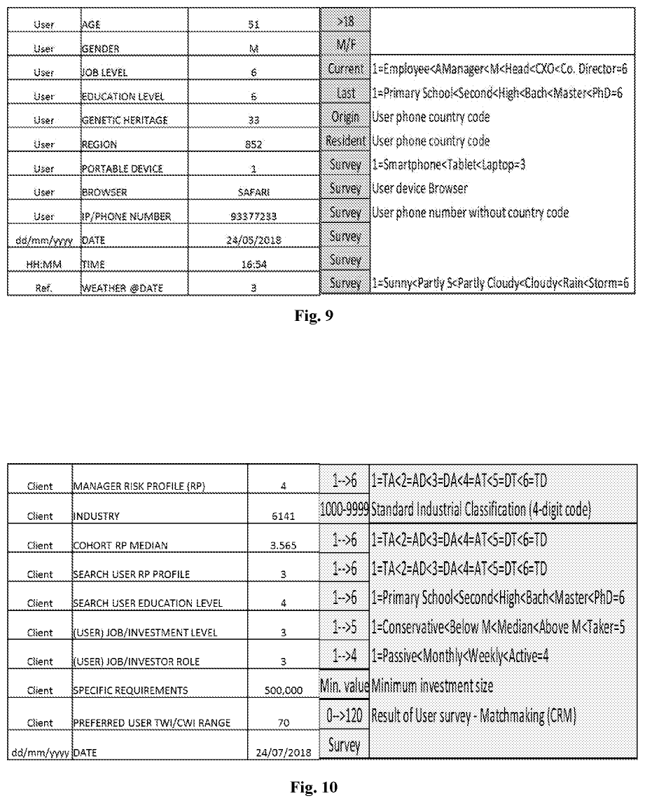

FIG. 9 is a table showing the demographic non-sensitive data and proxies collected at signup page for registration and an example of the test subject data shown in FIG. 8. It includes the basic information such as age, gender, job level, education level, and region, and more specific proxies used for profiling.

FIG. 10 is a table showing the data provided by a client who is ordering a search of a specific user profile such as the test subject shown in FIG. 8. The client data are also non-sensitive such as the client's relationship manager (RM) risk profile which requires this RM to take the test of this invention prior to ask for matchmaking with a specific user; the client industry in Standard Industrial Classification: the client cohort risk profile median which is reference to match the client expectation; the client search instructions for a specific user performance: user risk profile, user education level, user expected job/investment level, user expected job/investor role, specific requirements such as minimum investment size, university degree etc. and preferred user TWI or CWI range; date of the search. The relationships between the required information for the subject and the client and the neuropsychological performance testing process in reference to FIGS. 8, 9 and 10 are described in the Table 1.

FIG. 15 is a table of High Processing Performance & Flexibility of the present invention, which showing the 5 different pools of information collected by the devices in FIG. 2 and processed and aggregated during the entire process described in FIGS. 3 and 4 by the technology described in FIGS. 5 and 6. The purpose of detailing the performance and counting the number of datapoints collected and processed by the system of this invention is to demonstrate the density of information from which the invention derives its results. The user data and proxies account for 12 datapoints, the client search information accounts for 10 datapoints, the questionnaire answers account for 30 datapoints (only valid), the average latency and analysis of the timing graph accounts for 5 datapoints and finally the emotions capture account for 30 (1 per question, only valid answer) plus 5 facial expression analysis equals 35 datapoints. The total amount of datapoints generated and processed during the survey is 12×10×5×35×30 equals 630,000 datapoints. And for each additional question that can be recorded during subsequent uses of the neuropsychological performance test, there will be an accrual of 12×10×5×6×1 equals 3,600 data points. From a technical or a commercial point of view, the use of artificial intelligence analysis and statistical analysis combined to obtain such density of datapoints on a single individual marks a step towards a more unbiased approach to personality and identity that not only add credibility to the profile but allows rational decision driven by quantitative guidance whether on the user or the client side.

FIG. 11 is an illustration of the various parameters of the present invention. Including, the Risk Profile (RP) is a resulting combination of the primary and secondary temperament scores, the proportion of Yes versus No answers defines Decidership type (D) whether positive or negative—and is delivered in the percentage format as obtained by the highest of the 2 possibilities, the Truthfulness (T) is the ratio of the number of questions divided by this number plus the number of eventual revalidation multiplied by 100, the average latency of the test subject is divided by the latency median of the reference population that gives away the Thinking Type (TT) score, the Leadership (LS) score is the Thinking Type multiplied by the Job and Education levels, the Job Fitness (JF) score is the Leadership divided by the age in years, the Contradiction (CT) score is the Leadership multiplied by (1 minus the Decidership), the Biometric Type (BT) is the resulting sum of the averaged primary and secondary emotions coefficient divided by 2, and the confidence index (C) is calculated by the mobile AI processing the facial recognition at the front end of the invention.

FIG. 12 is a table showing the possible ranges of the scores delivered at the end of processing by the present invention. 3 types of Metrics are presented and correlated to build a tri-dimensional 3D profile of the test subject or user. The first part is called Psychometrics and refers to the psychological part of the performance test. The primary and secondary scores of the Risk Profile are given by the distribution of the answer's datapoints during the survey in the 3 dimensions/bins. The validity is determined by the number of revalidation (failed responses whether emotional or cognitive or timing) recorded during a single event test and within the limits, for example of 20%, or a maximum, for example of up to 6 additional questions. The Decidership range is 50-80% because the minimum of Yes or No answer should be 20%. The truthfulness range is 83-100% because 30/36 or 83% corresponds to the maximum of revalidation. Below that the test is deemed invalid. The second part is called Chronometric and refers to the timing part (using a chronometer) of the performance test. The range for the average user latency for a full test is between 2,000 and 7,000 milliseconds regardless the fact that some single question may record unusual latency during the 30 questions test. The Thinking Type range is 0.25 to 3.5. The Leadership score ranges between 0.25 and 125. The range for Job Fitness is between 0 to 7. The degree of contradiction can range between 0 and 62. The third part is called the Biometrics and refers to the result of facial emotions recognition part of the performance test. Whether the eyes, nose, lips, chin and other features of the face are accurately recognized and marked or not leads to a Confidence score in % which is delivered by the mobile AI regarding its ability to recognize the 8 types of emotion accurately. The Biometric Type refers to an average mean of the primary (Yes group, positive emotion) and secondary (No group, negative emotion) emotions and ranges between 1 to 4.

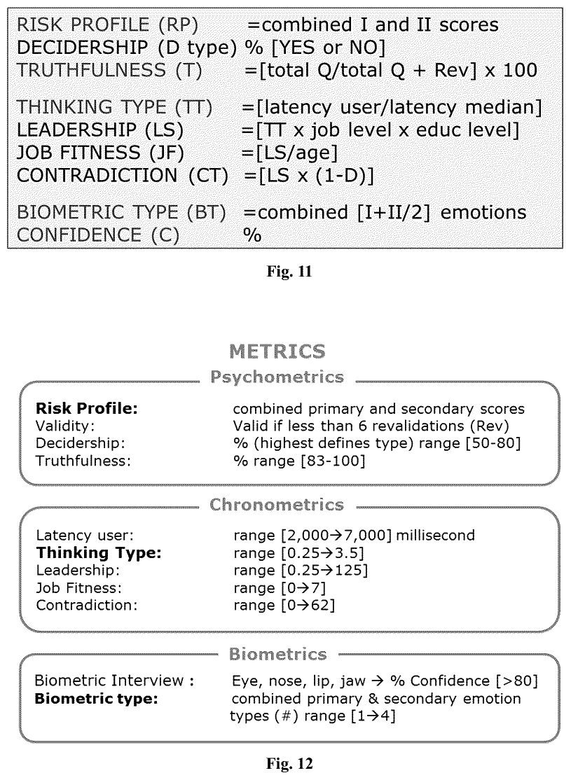

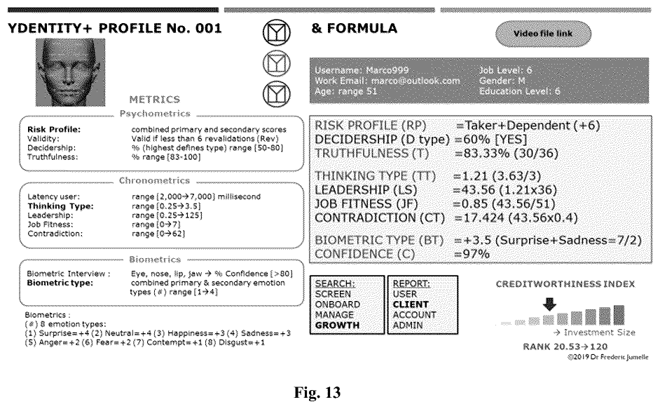

FIG. 13 is a table showing a comprehensive user performance of the scores obtained by the test subject described in FIGS. 8 and 9 using the present invention. In summary, the above-mentioned user has a RP type of Taker-Dependent which scores +6, a D score of 60% type YES, a T score of 83.33%, a TT score of 1.21, a LS score of 43.56, a JF score of 0.85, a BT type of +3.5 with a Confidence score of 97%. The user CWI index is 20.53 on a scale of 0 to 120. The profile was asked by a client in Financial Services Industry for upselling financial product i.e. the growth of investment portfolio. Unfortunately for the client, this user has received few low scores such as the T and his latency is close to median which could mean a slow brain activity for someone of this age (Male 51) or temporary issues or lack of concentration and that influences the results. The invention will recommend that a new test must be taken in a minimum of 6 to 12 months. The search type can be selected in the menu, for example: Screen, Onboard, Manage, Growth etc. And the type of report must be selected by the search ordering entity, for example: User, Client, Account, Admin, etc.

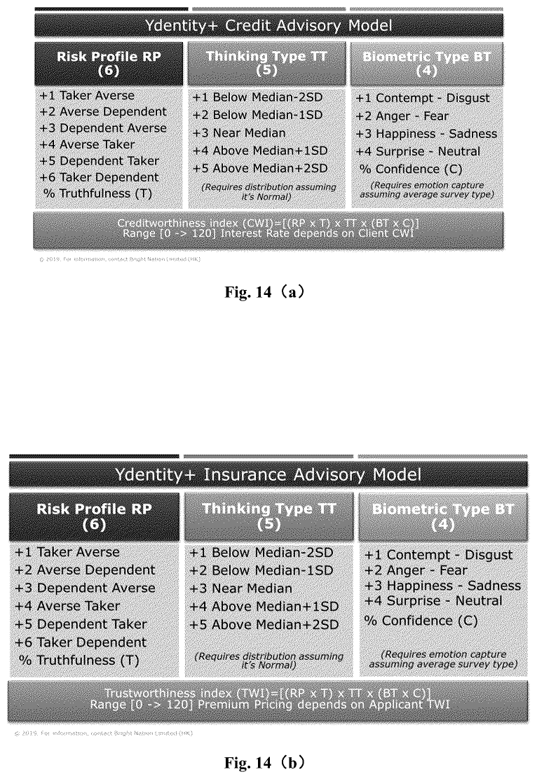

FIGS. 14(a) and 14(b) are table dedicated to explanatory analysis for advisory function using the performance test results of the present invention. The test results are divided into the similar three parts described in FIG. 12. The first part is the Risk Profile (RP) which is divided into 6 categories that have been attributed a coefficient according to the social interest of the risk scores combination (primary+secondary). For example, +1 for Taker Averse; +2 for Averse Dependent; +3 for Dependent Averse; +4 for Averse Taker; +5 for Dependent Taker; +6 for Taker Dependent. The second part is the Thinking Type (TT) which is divided into 5 categories according to the distribution of the population, variance and standard deviation, and subject to changes in sub-population and special cohort testing. Assuming that population is normal or quasi normal, the coefficient has been attributed according to the underlying thinking process of the test subject, the length of the brain processing is an indicator of the maturity of the brain. For example: +1 is for minus 2 standard deviations (−2SD) below median at the extreme low end of the population −5%; +2 is for minus 1 standard deviation (−1SD) below median usually between −5 and −15% of the population; +3 is for the nearby median of population usually 70% of the population between −15 and +15%; +4 is for plus 1 standard deviation (+1SD) above median usually between +15 and +5% of the population; +5 is for plus 2 standard deviations (+2SD) above median at the extreme high end of the population. The third part is the Biometric Type (BT) which is divided into 4 categories assuming that is the average survey type between highest positive (Yes answers) emotion score and highest negative (No answers) emotion score. The coefficient has been attributed to the emotions in virtue of the social interest and communication value of showing the average emotional reaction. For example: +1 is for Contempt or Disgust; +2 is for Anger or Fear; +3 is for Happiness or Sadness; +4 is for Surprise or Neutral. As a result of having these scores calculated, the final calculus of the indices whether CWI for Credit Advisory or TWI for Insurance Advisory is greatly simplified by the following formula:

$$CWI or TWI = [ ( RP \times T ) \times TT \times ( BT \times C ) ]$$

Therefore, the indices can range between 0 and 120. Maximum 120 is [6×5×4] assuming T=100% and C=100%.

In addition to the fields described above, the present invention can also be applied to pre-screening, remote screening and onboarding of human resources; sorting of personal accounts, fraud prevention and forensics for social media; matchmaking in client relation management and dating; onboarding and remote onboarding of new customers in the financial services industry in compliance to Know Your Client (“KYC”) or Customer Due Diligence (“CDD”) regulations and also any new virtual services to persons including providing smart ID for smart cities etc. Compatible with other identification and identity authentication/verification technologies, the present invention allows the creation of true personal identity by using personal metrics with a high degree of accuracy and security. So that, some of the biggest challenges of internet nowadays with an outrageous amount of fake accounts of which the fake social media accounts can pose a threat to society could also be dealt with through the present invention.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowcharts or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated. Accordingly, the enhanced assessment module supports cognitive and behavioral assessment of a participant subject in the field, and at the same time provides a unique employment of test and associated test batteries for the assessment.

It will be appreciated that, although specific embodiments of the invention have been described herein for purposes of illustration, various modifications may be made without departing from the spirit and scope of the invention. Accordingly, the scope of protection of this invention is limited only by the following claims and their equivalents.

1-26. (canceled)

27. A system for neuropsychological performance test, comprising:
A terminal device (101), used to interact with a cloud server (102) which stores user information and is logged into by user through the terminal device (101);
the user information obtained by the terminal device (101) are input and stored to the cloud server (102) in a login state;
a test module (400) comprises the user information, which is stored in the cloud server (102) or can be downloaded from the cloud server (102), and is directly accessed through said terminal device (101) and is trained by artificial neural network; and
said user information comprises user biometrics or emotions identification information, neuropsychological performance test answers information, and user latency or user chronometric information; and said terminal device (101) displays neuropsychological performance test results.

28. The system as claimed in claim 27, wherein said artificial neural network is a supervised, fully connected, feed-forward artificial neural network, using back-propagation training and generalized delta rule learning.

29. The system as claimed in claim 27, wherein the user biometrics or emotions identification information comprises facial expression information and/or voice information, wherein the facial expression information is obtained by an imaging device (108) and/or the voice information is obtained by a voice input device (109), and neuropsychological performance test answers are obtained by a questionnaire selection device (107) within a input device (106).

30. The system as claimed in claim 27, wherein the test module (400) comprises a questionnaire, which are generally YES or NO questions, in the number of 30 per questionnaire in order to obtain temperament dimensions information, answer of each question belongs to one of the temperament dimensions among HA/NS, RD/HA, NS/RD, NS/HA, HA/RD, RD/NS.

31. The system as claimed in claim 28, wherein said artificial neural network is composed of an input layer, a hidden layer, and an output layer, wherein said input layer consists of three nodes of question type, answer to the question, and emotions, respectively; said output layer consists of one node of risk profile; and the output of each node is:
$$𝒴 = \vec{w} x + \vec{b}$$ [symbol]
is the weight of this node [symbol]
is the bias.
The weights and bias with random values are initialized;
the activation function is applied to the output of the node. In this case ReLU is applied, given by:
$$ϕ ( a ) = max ( 0 , a ) = max ( 0 , \vec{w} x + \vec{b} )$$
The output of output layer is given by summing up the output of all nodes in the last layer:
$$𝒴 = ϕ ( {\vec{w}}_{0} \cdot ( {\sum }_{1}^{N} {\vec{w}}_{h} x + {\vec{b}}_{h} ) + {\vec{b}}_{0} )$$ [symbol]
ₕ is weight of last hidden layer [symbol]
ₕ is bias of last hidden layer [symbol]
₀ and [symbol] ₀ are weight and bias of output layer.

32. The system as claimed in claim 31, wherein the backpropagation is run to minimize the error between the validation datasets and the corresponding targets:
$${δ}_{h} = {𝒴}_{0} ( 1 - {𝒴}_{0} ) ( {𝒴}_{0} - t )$$ [symbol]
₀ is the output of output layer
t is the target output (ground truth)
For each node, calculate the back propagation error term:
$${δ}_{h}^{′} = {𝒴}_{h}^{′} ( 1 - {𝒴}_{h}^{′} ) {\sum }_{1}^{N} {δ}_{h} \cdot {\vec{w}}_{h}$$
Update the synaptic weights from a node in layer n to a node in layer n+1, given by:
$$Δ \vec{w} = - {𝒴δ}_{h}^{′} x$$ [symbol]
is learning rate, then:
$$\vec{w} \text{=:} \vec{w} + Δ \vec{w}$$
Calculate mean squared errors:
$$MSE = \frac{1}{{N}_{ts}} \cdot {\sum }_{1}^{{N}_{ts}} {( {𝒴}_{0} - t )}^{2}$$
15% of the data is used for validation dataset.

33. The system as claimed in 32, wherein repeat the forward propagation and back propagation until the number of the epoch limit or early stopping criteria is reached, and after training, the model runs on 15% test dataset to calculate precision, accuracy and F score.

34. The system as claimed in claim 28, wherein the excitation propagation phase comprises a propagation link in each iteration consists of two steps:
1). forward propagation phase: the training input is sent to the network to obtain the excitation response; and
2). back propagation phase: the excitation response corresponds to the training input.

35. The system as claimed in claim 34, wherein the output is evaluated to obtain a response error of the output layer and the hidden layer; in the weight update phase, for each junction between nodes weight, update consists of two steps:
1). Multiply the input stimulus and response error to obtain a gradient of weights;
2). Multiply this gradient by a scale and invert it added to the weight.

36. The system as claimed in claim 27, wherein the trustworthiness (TWI) and creditworthiness index (CWI) of the user can be obtained based on said neuropsychological performance test results according to the following formula:
$$Psychological trustworthiness index TWI = [ ( RP \times T ) \times TT \times ( BT \times C ) ] \qquad ( 1 )$$
RP is a Risk Profile, T is Truthfulness, TT is a Thinking Type, BT is a Biometric Type and C is a Confidence score; and the TWI or CWI is in the scope of 0-120.

37. A method for conducting neuropsychological performance test, comprising:
Using a terminal device (101) to interact with a cloud server (102) which stores user information and is logged into by user through the terminal device (101);
Inputting and storing the user information obtained by the terminal device (101) to the cloud server (102) in a login state;
Accessing a test module (400) comprising the user information directly through said terminal device (101), which is stored in the cloud server (102) or can be downloaded from the cloud server (102), and training said test module (400) by artificial neural network; and
said user information comprises user biometrics or emotions identification information, neuropsychological performance test answers information, and user latency or user chronometric information; and
displaying neuropsychological performance test results by said terminal device (101).

38. The method as claimed in claim 37, wherein said artificial neural network is a supervised, fully connected, feed-forward artificial neural network, with an excitation propagation phase, using back-propagation training and generalized delta rule learning.

39. The method as claimed in claim 37, wherein the user biometrics or emotions identification information comprises facial expression information and/or voice information, wherein the facial expression information is obtained by an imaging device (108) and/or the voice information is obtained by a voice input device (109), and neuropsychological performance test answers are obtained by a questionnaire selection device (107) within a input device (106).

40. The method as claimed in claim 37, wherein the test module (400) comprises a questionnaire, which are generally YES or NO questions, in the number of 30 per questionnaire in order to obtain temperament dimensions information, answer of each question belongs to one of the temperament dimensions among HA/NS, RD/HA, NS/RD, NS/HA, HA/RD, RD/NS leading to one or two emotions type among Contempt (“CO”); Anger (“AN”); Sadness (“SA”); Neutral (“NE”); Surprise (“SU”); Happiness (“HP”); Fear (“FE”); and Disgust (“DI”).

41. The method as claimed in claim 38, wherein said artificial neural network is composed of an input layer, a hidden layer, and an output layer, wherein said input layer consists of three nodes x₁, x₂, and x₃, x₁ is question type, x₂ is answer to the question, and x₃ is emotions, respectively; said output layer consists of one node of risk profile; and the output of each node is:
$$𝒴 = \vec{w} x + \vec{b}$$ [symbol]
is the weight of this node; and [symbol] is the bias, the weights and bias with random values are initialized;
the activation function is applied to the output of the node. In this case ReLU is applied, given by:
$$ϕ ( a ) = max ( 0 , a ) = max ( 0 , \vec{w} x + \vec{b} )$$
The output of output layer is given by summing up the output of all nodes in the last layer:
$$𝒴 = ϕ ( {\vec{w}}_{0} \cdot ( {\sum }_{1}^{N} {\vec{w}}_{h} x + {\vec{b}}_{h} ) + {\vec{b}}_{0} )$$ [symbol]
ₕ is weight of last hidden layer [symbol]
ₕ is bias of last hidden layer [symbol]
₀ and [symbol] ₀ are weight and bias of output layer.

42. The method as claimed in claim 41, wherein the backpropagation is run to minimize the error between the validation datasets and the corresponding targets:
$${δ}_{h} = {𝒴}_{0} ( 1 - {𝒴}_{0} ) ( {𝒴}_{0} - t )$$ [symbol]
₀ is the output of output layer
t is the target output (ground truth)
For each node, calculate the back propagation error term:
$${δ}_{h}^{′} = {𝒴}_{h}^{′} ( 1 - {𝒴}_{h}^{′} ) {\sum }_{1}^{N} {δ}_{h} \cdot {\vec{w}}_{h}$$
Update the synaptic weights from a node in layer n to a node in layer n+1, given by:
$$Δ \vec{w} = - {𝒴δ}_{h}^{′} x$$ [symbol]
is learning rate, then:
$$\vec{w} \text{=:} \vec{w} + Δ \vec{w}$$
Calculate mean squared errors:
$$MSE = \frac{1}{{N}_{ts}} \cdot {\sum }_{1}^{{N}_{ts}} {( {𝒴}_{0} - t )}^{2}$$
15% of the data is used for validation dataset.

43. The method as claimed in claim 42, wherein
repeat the forward propagation and back propagation until the number of the epoch limit or early stopping criteria is reached, and after training, the model runs on 15% test dataset to calculate precision, accuracy and F score.

44. The method as claimed in claim 43, wherein the excitation propagation phase comprises a propagation link in each iteration consists of two steps:
1). forward propagation phase: the training input is sent to the network to obtain the excitation response; and
2). back propagation phase: the excitation response corresponds to the training input.

45. The method as claimed in claim 44, wherein the output is evaluated to obtain a response error of the output layer and the hidden layer; in the weight update phase, for each junction between nodes weight, update consists of two steps:
1). Multiply the input stimulus and response error to obtain a gradient of weights;
2). Multiply this gradient by a scale and invert it added to the weight.

46. The method as claimed in claim 37, wherein the trustworthiness (TWI) and creditworthiness index (CWI) of the user can be obtained based on said neuropsychological performance test results according to the following formula:
$$Psychological trustworthiness index TWI = [ ( RP \times T ) \times TT \times ( BT \times C ) ] \qquad ( 1 )$$
RP is a Risk Profile, T is Truthfulness, TT is a Thinking Type, BT is a Biometric Type and C is a Confidence score; and the TWI or CWI is in the scope of 0-120.
